(19) United States
(12) Patent Application Publication
(10) Pub. No.: US 2020/0147353 A1
(43) Pub. Date: May 14, 2020

(54) WIRE FOR MEDICAL TREATMENT INSTRUMENT AND GUIDE WIRE

(71) Applicant: TOKUSEN KOGYO CO., LTD., Ono-shi, Hyogo (JP)

(72) Inventors: Masaomi TACHIBANA, Kakogawa-shi, Hyogo (JP); Shinji MIYAMOTO, Kakogawa-shi, Hyogo (JP)

(73) Assignee: TOKUSEN KOGYO CO., LTD., Ono-shi, Hyogo (JP)

(21) Appl. No.: 16/630,882

(22) PCT Filed: Jun. 26, 2018
(86) PCT No.: PCT/JP2018/024237

§ 371 Date: Jan. 14, 2020

(30) Foreign Application Priority Data
Jul. 20, 2017 (JP) 2017-140985

(51) Int. Cl. A61M 25/09; A61L 31/02; A61L 31/14; B21C 1/02

(52) U.S. Cl. CPC A61M 25/09; A61L 31/022; C22C 38/40; B21C 1/02; A61M 2025/09075; A61L 31/14

(57) ABSTRACT

A contour of a cross-section perpendicular to a longitudinal direction of a wire 2 is a circle having a diameter of D. An imaginary circle 4 which is concentric with the circle of the contour of the wire 2 and has a diameter that is ¾ of the diameter D, is assumed. On the imaginary circle 4, a first measurement point M1, a second measurement point M2, a third measurement point M3, a fourth measurement point M4, a fifth measurement point M5, a sixth measurement point M6, a seventh measurement point M7, and an eighth measurement point M8 are assumed. A Vickers hardness (Hv) is measured at each of the eight measurement points. A standard deviation σ of the eight measurement values is not greater than 10. The average of the eight measurement values is preferably not less than 670 and preferably not greater than 770.

### TECHNICAL FIELD

The present invention relates to wires suitable for medical treatment instruments and guide wires having cores which are obtained from the wires.

### BACKGROUND ART

In tests and treatments using catheters, a guide wire is inserted in a blood vessel. The catheter is inserted in the blood vessel along the guide wire. The catheter is moved into the blood vessel while being guided by the guide wire. After the leading end of the catheter has reached a predetermined position, the guide wire is removed from the blood vessel. A contrast medium or the like is administered through the catheter.

The guide wire has a core and a cover that covers the core. The guide wire is used for a human body, and, therefore, the core needs to be corrosion-resistant. Austenitic stainless steel is preferably used for the core.

The blood vessel is bent, and, therefore, the guide wire inserted in the blood vessel is moved thereinto while being bent. A doctor may repeatedly move the guide wire forward and backward in order to cause the guide wire to pass through a narrow portion. The breakage of the core needs to be prevented even in such a usage. In other words, the core needs to be fatigue-resistant.

A doctor operates a portion, of the guide wire, which is located outside the human body, in a state where the guide wire is inserted in a blood vessel. In this operation, the doctor rotates the guide wire. The torque of the rotation is transmitted to the leading end of the guide wire. The core needs to have torque transmittability.

An example of the guide wire having excellent fatigue resistance and torque transmittability is disclosed in JP2009-172229.

### CITATION LIST

### Patent Literature

Patent Literature 1: JP2009-172229

### SUMMARY OF THE INVENTION

### Problems to be Solved by the Invention

A human body has a portion in which a blood vessel is bent at a steep angle. A high bending stress is applied to the core of the guide wire that passes through the portion. Therefore, a fatigue resistance needs to be increased for the core of the guide wire that passes through the portion. Thus, a wire as a material of the core also needs to have a high fatigue resistance.

A wire used in this medical treatment instrument may be required to have a high fatigue resistance, also when used for various medical treatment instruments other than the guide wire.

An object of the present invention is to provide a wire, for medical treatment instruments, having extremely excellent fatigue resistance.

### Solution to the Problems

In a wire, for a medical treatment instrument, according to the present invention, a contour of a cross-section perpendicular to a longitudinal direction is a circle having a diameter of D. On the cross-section, a standard deviation σ of Vickers hardnesses at eight measurement points that are equally spaced from each other on an imaginary circle which is concentric with the circle and has a diameter of (¾)D, is not greater than 10.

Preferably, an average of the Vickers hardnesses at the eight measurement points is not less than 670 and not greater than 770.

Preferably, a material of the wire for a medical treatment instrument is a stainless steel.

Preferably, a tensile strength of the wire for a medical treatment instrument is not less than 2600 MPa.

Preferably, a straightness of the wire for a medical treatment instrument is not greater than 0.10 mm when a length of the wire is 2.00 m.

From another viewpoint, a guide wire according to the present invention has a core. A contour of the core on a cross-section perpendicular to a longitudinal direction is a circle having a diameter of D. On the cross-section, a standard deviation σ of Vickers hardnesses at eight measurement points that are equally spaced from each other on an imaginary circle which is concentric with the circle and has a diameter of (¾)D, is not greater than 10.

Preferably, an average of the Vickers hardnesses at the eight measurement points is not less than 670 and not greater than 770.

Preferably, a material of the core is a stainless steel.

Preferably, a tensile strength of the core is not less than 2600 MPa.

### Advantageous Effects of the Invention

The inventor of the present invention has found that the wire for a medical treatment instrument is broken due to concentration of stress. The inventor of the present invention has found that the concentration of the stress is caused by variation in hardness in the circumferential direction of the wire. In the wire, for a medical treatment instrument, according to the present invention, the standard deviation σ of the Vickers hardnesses is small. The wire has an excellent fatigue resistance.

### BRIEF DESCRIPTION OF THE DRAWINGS

FIG. 1 is a perspective view of a part of a wire, for a medical treatment instrument, according to one embodiment of the present invention.

FIG. 2 is an enlarged cross-sectional view of the wire shown in FIG. 1.

FIG. 3 is a front view of the wire shown in FIG. 1.

FIG. 4 is a conceptual diagram illustrating a production device for the wire shown in FIG. 1.

FIG. 5 is a plan view of a first corrective unit of the device shown in FIG. 4.

FIG. 6 is an enlarged front view of a corrective roller of the first corrective unit shown in FIG. 5.

FIG. 7 illustrates measurement of straightness of the wire shown in FIG. 1.

FIG. 8 is a cross-sectional view of a part of a guide wire according to one embodiment of the present invention.

FIG. 9 is a front view of a core of the guide wire shown in FIG. 8.

### DESCRIPTION OF EMBODIMENTS

The following will describe in detail the present invention based on preferred embodiments with reference where appropriate to the accompanying drawing.

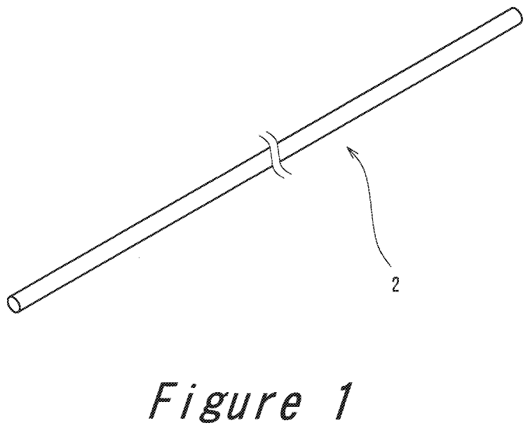

FIG. 1 illustrates a wire 2 for a medical treatment instrument. The wire 2 has a long shape. The thickness of the wire 2 is typically not greater than 2.0 mm, and, in particular, not greater than 1.0 mm. The material of the wire 2 is a metal.

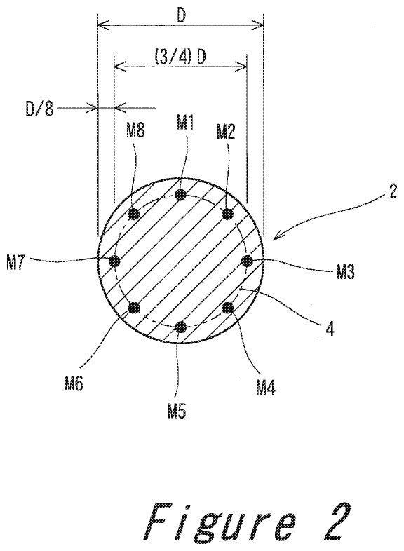

FIG. 2 illustrates a cross-section of the wire 2. The cross-section is perpendicular to the longitudinal direction of the wire 2. As is apparent from FIG. 2, the cross-section has a circular contour. The contour need not be a perfect circle. In the present invention, the contour which is slightly different from a perfect circle due to an error in production or the like is also called “circle”.

In FIG. 2, the diameter of the circle of the contour is indicated by an arrow D. In other words, the wire 2 has a diameter of D. In FIG. 2, an alternate long and two short dashes line indicated by reference numeral 4 represents an imaginary circle. The imaginary circle is concentric with the circle of the contour of the wire 2. A ratio of the diameter of the imaginary circle 4 to the diameter D is ¾. Therefore, a distance from the surface of the wire 2 to the imaginary circle 4 is D/8 as shown in FIG. 2.

A first measurement point M1 is assumed on the imaginary circle 4. The position of the first measurement point M1 is randomly determined. Next, a second measurement point M2 is assumed, on the imaginary circle 4, at a position distant from the first measurement point M1 by 45° as the central angle of the imaginary circle 4. Similarly, a third measurement point M3, a fourth measurement point M4, a fifth measurement point M5, a sixth measurement point M6, a seventh measurement point M7, and an eighth measurement point M8 are assumed in increments of 45°. These eight measurement points are disposed on the imaginary circle 4 at equal pitch angles.

For each of the eight measurement points, a Vickers hardness (Hv) is measured. The Vickers hardness is measured by using a micro Vickers hardness tester in compliance with “JIS Z 2244:2009”. The measurement condition is as follows.

Temperature: 23° C.

Load: 100 gf

Since the number of the measurement points is eight, eight measurement values (Vickers hardnesses) are obtained. A standard deviation σ of these measurement values is calculated. The standard deviation σ is preferably not greater than 10. In the wire 2, for a medical treatment instrument, in which the standard deviation σ is not greater than 10, concentration of stress in the circumferential direction is inhibited. The wire 2 has an excellent fatigue resistance. The wire 2 is not easily broken when used for a human body. From this viewpoint, the standard deviation σ is more preferably not greater than 8 and particularly preferably not greater than 5. Idealistically, the standard deviation σ is zero.

The average Av of these eight measurement values (Vickers hardnesses) is preferably not less than 670 and preferably not greater than 770. The wire 2, for a medical treatment instrument, for which the average Av is not less than 670 has excellent torque transmittability. From this viewpoint, the average Av of the Vickers hardnesses is more preferably not less than 690 and particularly preferably not less than 700. The wire 2 for which the average Av is not greater than 770 is not brittle. Therefore, the wire 2 is not easily broken. From this viewpoint, the average Av is more preferably not greater than 750 and particularly preferably not greater than 740.

A ratio of the standard deviation σ to the average Av of the Vickers hardnesses is preferably not greater than 2.0%. In the wire 2, for a medical treatment instrument, for which the ratio is not greater than 2.0%, concentration of stress in the circumferential direction is inhibited. The wire 2 has an excellent fatigue resistance. The wire 2 is not easily broken when used for a human body. From this viewpoint, the ratio is more preferably not greater than 1.5% and particularly preferably not greater than 0.7%. Idealistically, the ratio is zero.

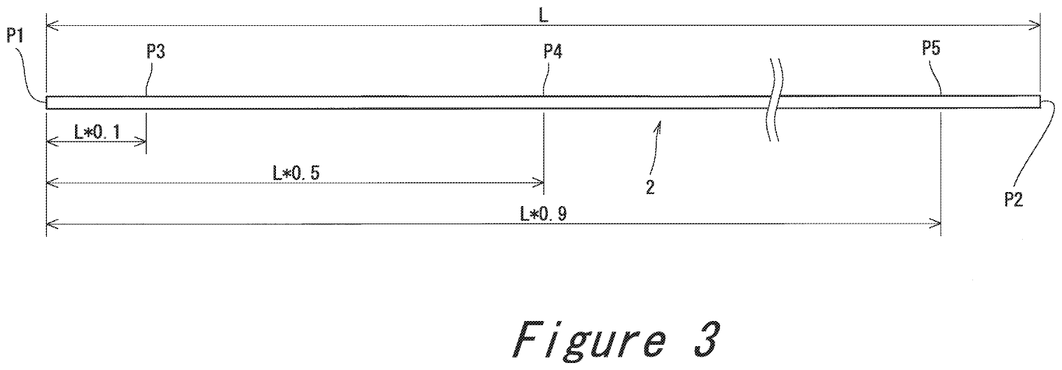

FIG. 3 is a front view of the wire 2, for a medical treatment instrument, shown in FIG. 1. In FIG. 3, an arrow L represents the entire length of the wire 2. The entire length L is a distance from a front end P1 to a rear end P2. In FIG. 3, reference numeral P3 represents a point at which a distance from the front end P1 is L*0.1, reference numeral P4 represents a point at which a distance from the front end P1 is L*0.5, and reference numeral P5 represents a point at which a distance from the front end P1 is L*0.9.

At the point P3, a first cross-section is obtained by the wire 2 being cut. The first cross-section is perpendicular to the length direction of the wire 2. On the first cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. At the first cross-section, the standard deviation σ, the average Av, and the ratio (o/Av) are achieved within the above-described ranges.

At the point P4, a second cross-section is obtained by the wire 2 being cut. The second cross-section is perpendicular to the length direction of the wire 2. On the second cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. Also at the second cross-section, similarly to the first cross-section, the standard deviation σ, the average Av, and the ratio (σ/Av) are achieved within the above-described ranges.

At the point P5, a third cross-section is obtained by the wire 2 being cut. The third cross-section is perpendicular to the length direction of the wire 2. On the third cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. Also at the third cross-section, similarly to the first cross-section, the standard deviation σ, the average Av, and the ratio (σ/Av) are achieved within the above-described ranges.

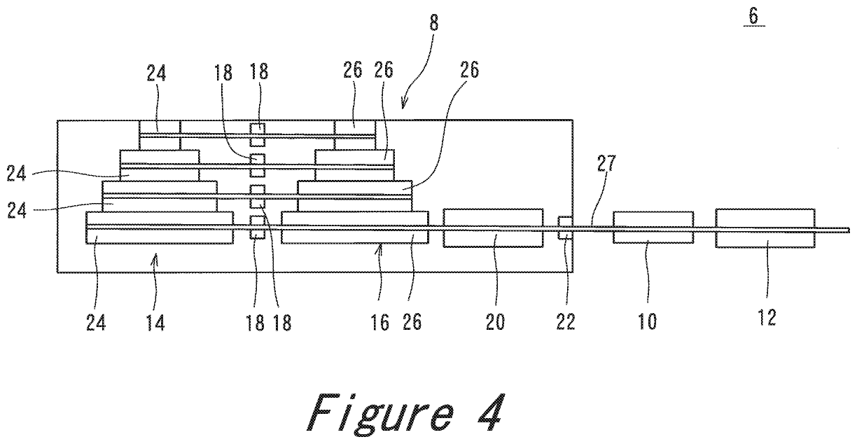

FIG. 4 is a conceptual diagram illustrating a production device 6 for the wire shown in FIG. 1. The device 6 includes a wire drawing machine 8, a drawn-wire take-up machine 10, and a second corrective unit 12. The wire drawing machine 8 has a first cone 14, a second cone 16, a plurality of dies 18, a first corrective unit 20, and a final die 22. The first cone 14 has a plurality of rollers 24 having different diameters. The second cone 16 also has a plurality of rollers 26 having different diameters. A base wire 27 is extended on and between the first cone 14 and the second cone 16. The base wire 27 passes through the dies 18 while being moved from the first cone 14 to the second cone 16. The base wire 27 is moved from the rollers 24 and 26 having smaller diameters to the rollers 24 and 26 having larger diameters. By the movement, the base wire 27 is elongated, and has reduced diameter. The base wire 27 passes through the first corrective unit 22, the final die 22, the drawn-wire take-up machine 10, and the second corrective unit 12.

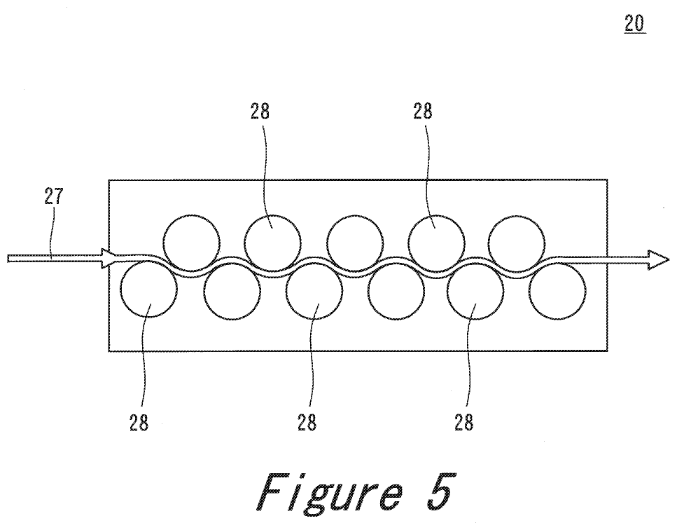

FIG. 5 is a conceptual diagram illustrating the first corrective unit 20 of the device 6 shown in FIG. 4. A not-illustrated structure of the second corrective unit 12 is the same as the structure of the first corrective unit 20. The first corrective unit 20 has a plurality of corrective rollers 28 that are disposed so as to zigzag. In the embodiment in FIG. 5, the number of the corrective rollers 28 is 11.

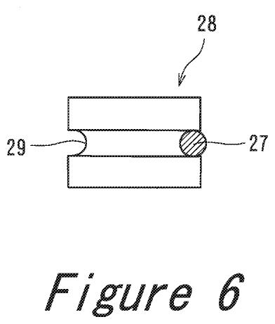

FIG. 6 is an enlarged front view of each corrective roller 28 of the first corrective unit 20 shown in FIG. 5. The corrective roller has corrective grooves 29. The width of each corrective groove 29 is almost the same as the diameter of the base wire 27. In the embodiment in FIG. 5, the number of the corrective rollers 28 is 11. The base wire 27 is moved so as to zigzag along the corrective rollers 28. For the base wire 27, a repeated bending process is performed over the entirety of the surface portion by the corrective grooves 29. Thus, uniformity of the hardness over the surface portion is increased.

After the process by the device 6 shown in FIGS. 4 and 5, the base wire 27 is cut so as to have a predetermined length, and is further subjected to heat treatment, to obtain the wire 2 for a medical treatment instrument. The wire drawing condition is adjusted well, thereby obtaining the wire 2 having a small standard deviation σ. The inventor of the present invention has found that the wire 2 having a small standard deviation σ can be obtained by setting the final wire drawing condition as follows.

The number of corrective units: 2

Positions at which the corrective units are mounted: preceding and following the final die

The number of the corrective rollers: 9 to 13 Tension of the base wire at the outlet of the corrective machine: 40% to 70% of breaking load

Preferably, the heat treatment is performed in a hydrogen atmosphere. In the heat treatment in this atmosphere, heat is transmitted to the base wire in a short time. The temperature for the heat treatment is 500° C. to 650° C.

The material of the wire 2 is preferably a stainless steel. A stainless steel has excellent corrosion resistance and strength. Specific examples of the stainless steel include austenitic stainless steels, ferritic stainless steels, martensitic stainless steels, precipitation hardening stainless steels, and duplex stainless steels. The austenitic stainless steel is preferably used. Other preferable materials of the wire 2 are Ni—Ti alloys and Ti alloys.

A tensile strength of the wire 2 is preferably not less than 2600 MPa. The wire 2 having the tensile strength of not less than 2600 MPa has an excellent pushability when the wire 2 is moved into a human body. From this viewpoint, the tensile strength is more preferably not less than 2700 MPa and particularly preferably not less than 2800 MPa. The tensile strength is preferably not greater than 3000 MPa.

The tensile strength is measured in compliance with “JIS Z 2241 (2011)”. The measurement condition is as follows.

Temperature: 23° C.

Tensile rate: 10 mm/min

Distance between evaluation points: 100 mm

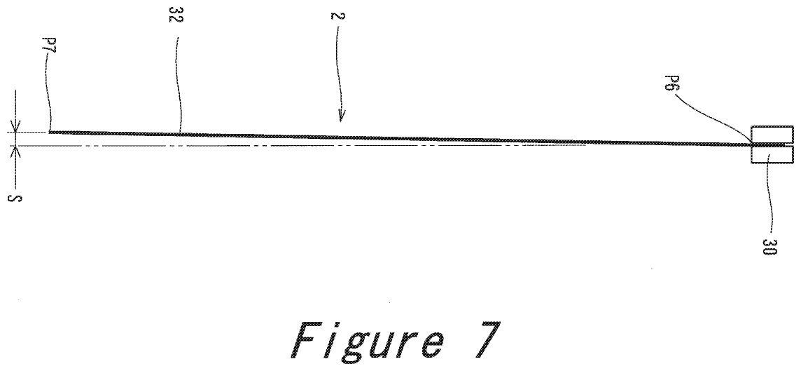

FIG. 7 illustrates measurement of straightness of the wire 2 shown in FIG. 1. In this measurement, a portion, of the wire 2, near the upper end is chucked by a tool 30. An unchucked portion of the wire 2 is called a free portion 32. Force acting on the free portion 32 is only gravity. In FIG. 7, a point P6 represents an upper end of the free portion 32, and a point P7 represents a lower end of the free portion 32. The distance from the upper end P6 to the lower end P7 is 2.00 m. In FIG. 7, an alternate long and two short dashes line extends in the vertical direction. In FIG. 7, reference numeral S represents a distance (mm) between the lower end P7 and the alternate long and two short dashes line. The distance S represents a deviation of the lower end P7 of the wire 8 from the vertical direction. The distance S represents the straightness. The wire 8 for which the distance S is small has an excellent straightness. For a wire having a poor straightness, the distance S has a great value due to the bending of the wire.

The straightness S of the wire 2 is preferably not greater than 0.10 mm. The wire 2 for which the straightness S is not greater than 0.10 mm has excellent torque transmittability. From this viewpoint, the straightness S is more preferably not greater than 0.05 mm and particularly preferably not greater than 0.02 mm. Idealistically, the straightness S is zero.

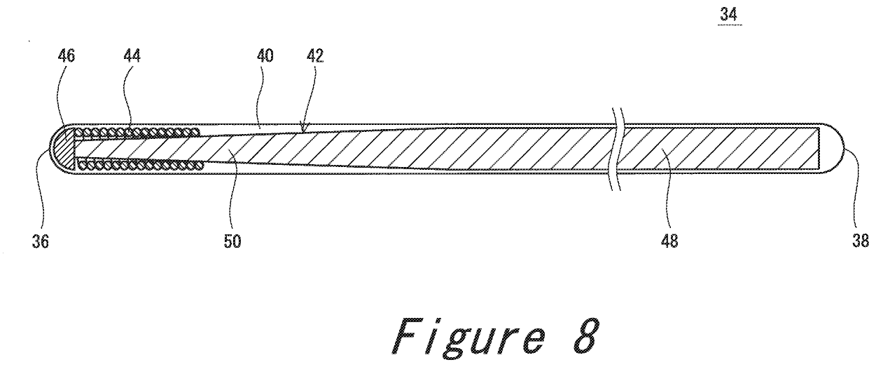

FIG. 8 is a cross-sectional view of a part of a guide wire 34 according to one embodiment of the present invention. In FIG. 8, the left end represents a leading end 36, and the right end represents a rear end 38. The guide wire 34 has a cover 40, a core 42, a coil 44, and a binder 46. The entire length of the guide wire 34 is typically 1500 mm to 2300 mm. The wire diameter (thickness) of the guide wire 34 is typically 0.30 mm to 0.60 mm.

The cover 40 covers the core 42. The cover 40 is formed from a synthetic resin. The synthetic resin is typically Teflon resin. The cover 40 allows achievement of smoothness for inserting the guide wire 34 in a blood vessel.

The core 42 includes a main portion 48 and a tapered portion 50. The wire diameter is substantially uniform in the main portion 48. In the main portion 48, the wire diameter is typically 0.25 mm to 0.50 mm. The diameter of the tapered portion 50 decreases toward the leading end 36.

The coil 44 is wound around the tapered portion 50. The coil 44 reinforces the tapered portion 50 without reducing flexibility of the tapered portion 50. The binder 46 is fixed to the core 42.

The core 42 is formed from the wire 2, for a medical treatment instrument, shown in FIGS. 1 to 3. The core 42 is formed by grinding a portion of the wire 2 near the leading end 36. Typically, the wire 2 is ground by a centerless grinding machine. The tapered portion 50 is formed by the grinding.

As described above, the core 42 is formed from the wire 2, for a medical treatment instrument, shown in FIGS. 1 to 3. Therefore, the core 42 has the same standard deviation σ, the average Av, and the ratio (σ/Av) of the Vickers hardnesses as the wire 2 for a medical treatment instrument. Furthermore, the material, the tensile strength, and the straightness S of the core 42 are the same as those of the wire 2 for a medical treatment instrument. Therefore, the core 42 has excellent fatigue resistance and torque transmittability. Also when a doctor repeats forward and backward movement of the guide wire 34 in a state where the guide wire 34 is inserted in a bent portion of a blood vessel, the core 42 is not easily broken. When the doctor rotates a portion, of the guide wire 34, near the rear end 38, the torque is transmitted to the leading end 36. Accordingly, the doctor is allowed to smoothly operate the guide wire 34.

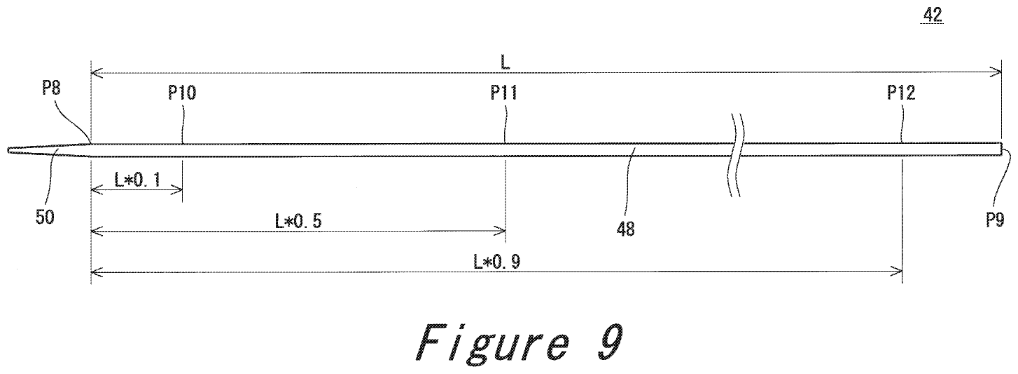

Since the core 42 has the tapered portion 50, the Vickers hardness cannot be measured in the method shown in FIG. 3. FIG. 9 shows a measurement method that can replace the method shown in FIG. 3. In FIG. 9, reference numeral P8 represents a boundary between the main portion 48 and the tapered portion 50, and an arrow L represents the entire length of the main portion 48. The entire length L is a distance from the boundary P8 to the rear end P9. In FIG. 9, reference numeral P10 represents a point at which a distance from the boundary P8 is L*0.1, reference numeral P11 represents a point at which a distance from the boundary P8 is L*0.5, and reference numeral P12 represents a point at which a distance from the boundary P8 is L*0.9.

At the point P10, the core 42 is cut to obtain a first cross-section. The first cross-section is perpendicular to the length direction of the core 42. On the first cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. At the first cross-section, the standard deviation σ, the average Av, and the ratio of the standard deviation σ to the average Av are achieved within the above-described ranges.

At the point P11, the core 42 is cut to obtain a second cross-section. The second cross-section is perpendicular to the length direction of the core 42. On the second cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. Also at the second cross-section, similarly to the first cross-section, the standard deviation σ, the average Av, and the ratio of the standard deviation σ to the average Av are achieved within the above-described ranges.

At the point P12, the core 42 is cut to obtain a third cross-section. The third cross-section is perpendicular to the length direction of the core 42. On the third cross-section, the first measurement point M1, the second measurement point M2, the third measurement point M3, the fourth measurement point M4, the fifth measurement point M5, the sixth measurement point M6, the seventh measurement point M7, and the eighth measurement point M8 are assumed as described above. At each of these measurement points, the Vickers hardness is measured. Also at the third cross-section, similarly to the first cross-section, the standard deviation σ, the average Av, and the ratio of the standard deviation σ to the average Av are achieved within the above-described ranges.

### EXAMPLES

The following will show the effects of the present invention by means of examples, but the present invention should not be construed in a limited manner based on the description of these examples.

### Example 1

A base wire formed from SUS304 as a material was repeatedly subjected to wire drawing and heat treatment. By the wire drawing, the base wire was elongated while the diameter of the base wire was being reduced. In the final wire drawing process step, a corrective process was performed by corrective units disposed preceding and following a finishing die. In the final wire drawing process step, the wire diameter was 0.35 mm. The condition for the correction was as follows.

Diameter of corrective roller: 10 mm

The number of corrective rollers: 11

Tension of base wire at corrective unit:
190 N (inlet of finishing die)
170 N (outlet of finishing die)

The base wire having been subjected to the final wire drawing was annealed at a low temperature, to obtain a core for a guide wire. The condition for the low temperature annealing was as follows.
Atmospheric temperature: 575° C.
Retention time: 60 min
Atmospheric gas: hydrogen

### Example 2

A core of example 2 was obtained in the same manner as in example 1 except that the number of corrective rollers at a corrective unit was as follows.

The number of corrective rollers: 9

### Example 3

A core of example 3 was obtained in the same manner as in example 1 except that tension of a base wire at a corrective unit was as follows.

Tension of base wire at corrective unit:
175 N (inlet of finishing die)
155 N (outlet of finishing die)

### Example 4

A core of example 4 was obtained in the same manner as in example 3 except that the number of corrective rollers at a corrective unit was as follows.

The number of corrective rollers: 9

### Comparative Example 1

A core of comparative example 1 was obtained in the same manner as in example 1 except that no corrective units were used in the final wire drawing process step.

### Comparative Example 2

A core of comparative example 2 was obtained in the same manner as in example 1 except that the number of corrective rollers and the tension of a base wire at a corrective unit were as follows.

The number of corrective rollers: 18

Tension of base wire at corrective unit:
190 N (inlet of finishing die)
190 N (outlet of finishing die)

[Evaluation]

The average value and the standard deviation of the cross-section hardnesses were measured in the above-described method. The tensile strength and the straightness of each core were measured in the above-described method. Furthermore, the fatigue value of each core was measured. The fatigue value was measured by using a Hunter fatigue tester manufactured by BEKAERT. When a test stress was 1000 to 1500 MPa in the atmosphere in which the humidity was 40%, a stress at which all of five test samples reached a fatigue limit of 10⁷ was set as a fatigue value. The results are indicated below in Table 1.

TABLE 1
Evaluation results

| | | | | | Comp. | Comp. |
|---|---|---|---|---|---|---|
| | Ex. 1 | Ex. 2 | Ex. 3 | Ex. 4 | Ex. 1 | Ex. 2 |
| Average hardness Av | 716 | 733 | 740 | 752 | 790 | 667 |
| (Hv 100 gf) | | | | | | |
| Hardness standard | 2.9 | 4.8 | 7.7 | 9.6 | 29.7 | 13.4 |
| deviation σ | | | | | | |
| σ/Av | 0.4 | 0.7 | 1.0 | 1.3 | 3.8 | 2.0 |
| Tensile strength (MPa) | 2709 | 2743 | 2758 | 2795 | 2872 | 2588 |
| Straightness (mm) | 0.01 | 0.01 | 0.01 | 0.02 | 0.10 | 0.10 |
| Fatigue value (MPa) | 1200 | 1175 | 1150 | 1125 | 1050 | 1100 |

The evaluation results in Table 1 clearly indicate that the present invention is superior.

### INDUSTRIAL APPLICABILITY

The wire according to the present invention is applicable to various medical treatment instruments.

### DESCRIPTION OF THE REFERENCE CHARACTERS

2 . . . wire for medical treatment instrument
4 . . . imaginary circle
6 . . . production device
8 . . . wire drawing machine
10 . . . drawn-wire take-up machine
12 . . . second corrective unit
14 . . . first cone
16 . . . second cone
18 . . . die
20 . . . first corrective unit
22 . . . final die
24, 26 . . . roller
27 . . . base wire
28 . . . corrective roller
29 . . . corrective groove
30 . . . tool
32 . . . free portion
34 . . . guide wire
40 . . . cover
42 . . . core
44 . . . coil
48 . . . main portion
50 . . . tapered portion

1: A wire for a medical treatment instrument, wherein
a contour of a cross-section perpendicular to a longitudinal direction is a circle having a diameter of D, and
on the cross-section, a standard deviation a of Vickers hardnesses at eight measurement points that are equally spaced from each other on an imaginary circle which is concentric with the circle and has a diameter of (¾)D, is not greater than 10.

2: The wire, for a medical treatment instrument, according to claim 1, wherein an average of the Vickers hardnesses at the eight measurement points is not less than 670 and not greater than 770.

3: The wire, for a medical treatment instrument, according to claim 1, wherein a material of the wire is a stainless steel.

4: The wire, for a medical treatment instrument, according to claim 1, wherein a tensile strength is not less than 2600 MPa.

5: The wire, for a medical treatment instrument, according to claim 1, wherein a straightness is not greater than 0.10 mm when a length of the wire is 2.00 m.

6: A guide wire comprising
a core, wherein
a contour of the core on a cross-section perpendicular to a longitudinal direction is a circle having a diameter of D, and
on the cross-section, a standard deviation σ of Vickers hardnesses at eight measurement points that are equally spaced from each other on an imaginary circle which is concentric with the circle and has a diameter of (¾)D, is not greater than 10.

7: The guide wire according to claim 6, wherein an average of the Vickers hardnesses at the eight measurement points is not less than 670 and not greater than 770.

8: The guide wire according to claim 6, wherein a material of the core is a stainless steel.

9: The guide wire according to claim 6 wherein a tensile strength of the core is not less than 2600 MPa.
